(19) United States
(12) Patent Application Publication
(10) Pub. No.: US 2024/0066251 A1
(43) Pub. Date: Feb. 29, 2024

(54) HEADBANDS, STRAPS, TABS, AND OTHER COMPONENTS OF AIR FLOW SYSTEMS

(71) Applicant: INVENT MEDICAL CORPORATION, Carlsbad, CA (US)

(72) Inventors: Nhien NGUYEN, Vista, CA (US); Steve HAN, Fountain Valley, CA (US); Samuel CHANG, Carlsbad, CA (US)

(21) Appl. No.: 18/237,307

(22) Filed: Aug. 23, 2023

Related U.S. Application Data
(60) Provisional application No. 63/400,335, filed on Aug. 23, 2022.
(60) Provisional application No. 63/400,336, filed on Aug. 23, 2022.
(60) Provisional application No. 63/400,337, filed on Aug. 23, 2022.
(60) Provisional application No. 63/400,340, filed on Aug. 23, 2022.
(60) Provisional application No. 63/400,341, filed on Aug. 23, 2022.
(60) Provisional application No. 63/466,537, filed on May 15, 2023.

(51) Int. Cl. A61M 16/06; A61M 16/08

(52) U.S. Cl. CPC A61M 16/0683; A61M 16/0672; A61M 16/0816; A61M 2205/3606

(57) ABSTRACT

A patient interface assembly for a head of a wearer is provided, comprising a nasal cannula or other patient interface, a first connector extending from a first side of the patient interface, a second connector extending from a second side of the patient interface, and a headband configured to wrap around at least a portion of the head of the wearer and maintain a position of the nasal cannula, a first strap coupled to a first strap coupling portion of the headband and configured to couple to the first connector, and a second strap coupled to a second strap coupling portion of the headband and configured to couple to the second connector, wherein the first strap is movable along the first strap coupling portion between the first stop and the second stop. A nasal cannula is provided comprising flexible prongs, and optionally a flexible tubular base.

### CROSS-REFERENCE TO RELATED APPLICATIONS

This application claims priority to U.S. Provisional Patent Application No. 63/400,335, filed Aug. 23, 2022, U.S. Provisional Patent Application No. 63/400,336, filed Aug. 23, 2022, U.S. Provisional Patent Application No. 63/400,337, filed Aug. 23, 2022, U.S. Provisional Patent Application No. 63/400,340, filed Aug. 23, 2022, U.S. Provisional Patent Application No. 63/400,341, filed Aug. 23, 2022, and U.S. Provisional Patent Application No. 63/466,537, filed May 15, 2023. These and all other extrinsic materials discussed herein, including publications, patent applications, and patents, are incorporated by reference in their entirety. Where a definition or use of a term in an incorporated reference is inconsistent or contrary to the definition of that term provided herein, the definition of that term provided herein applies and the definition of the term in the reference does not apply.

### TECHNICAL FIELD

The embodiments described herein are generally directed to headbands and other components of air flow systems.

### BACKGROUND

Some ventilators and other flow generation systems utilize a combination of single-use or reusable disposable components for the patient interface (e.g., mask or mouthpiece connected to flexible tubing), and non-disposable equipment such as air pumps, sensors, controller modules, and humidifiers, which can be used over a period of time with one or different users.

The patient interface can be for example a mouthpiece, mask (full face, nasal, pillow, total mask, or combinations of these), endotracheal tube, or tracheostomy tube. A cannula (e.g., a nasal cannula) often interfaces tubing that connects the ventilator or other flow generation device to a user, and therefore the air flow to the user's nose.

Standard nasal cannulas include a lightweight tube and two prongs that can be placed in the nostrils. The lightweight tube can be coupled to additional tubing. The cannula can be worn by a user by hooking the additional tubing around a patient's ears, or via certain types of headgear. However, known headgear devices are not readily adjustable, and are often made of uncomfortable materials such as hard or thick plastic that create pressure points, and are not easily adjustable—requiring removal or another person for adjustments. Further, known headgear tends to either easily slip off unless worn such that it digs into the top portions of a wearer's ears, which causes discomfort for users and a higher risk of undesirable removal.

### SUMMARY

The headbands, straps, connectors (e.g., tabs) and other components of air flow systems described herein advantageously overcome one or more of the problems identified above, and provide additional advantages over known air flow system components. These components are suitable for use with flow generation systems that generate a gaseous flow, for example airflow or a blend of ambient air and oxygen. A ventilator is one example of a flow generation system. A ventilator is a piece of medical equipment that delivers a flow of gas, such as a blend of oxygen and ambient air to the airway of a patient to assist in or substitute a patient's breathing. Most ventilators deliver a blend of oxygen and air so that the patient receives a target oxygen concentration greater than that of ambient air. Positive pressure therapy machines (e.g., CPAP) are also flow generation systems.

According to an aspect of the disclosure, a patient interface assembly for a head of a wearer (e.g., a patient or other user) is provided, comprising a nasal cannula (or a mask such as an oxygen face mask, a nasal pillow, etc.), a first connector extending from a first side of the nasal cannula (or a mask such as an oxygen face mask, a nasal pillow, etc.), a second connector extending from a second side of the nasal cannula (or a mask such as an oxygen face mask, a nasal pillow, etc.), a headband configured to wrap around at least a portion of the head of the wearer and maintain a position of the nasal cannula (or a mask such as an oxygen face mask, a nasal pillow, etc.), the headband comprising a first strap coupling portion and a second strap coupling portion, wherein the first strap coupling portion extends between a first stop and a second stop, a first strap having a first end portion and a second end portion, wherein the first end portion is coupled to the first strap coupling portion, wherein the first strap is movable along the first strap coupling portion between the first stop and the second stop (e.g., via a loop sized and dimensioned to receive at least a portion of the first strap coupling portion), and wherein the second end portion is coupled to the first connector (e.g., via a hook and loop fastener, or other fastener/fastening mechanism), and a second strap having a first end portion and a second end portion, wherein the first end portion is coupled to the second strap coupling portion, and wherein the second end portion is coupled to the second connector.

In some embodiments, the headband comprises a first end portion and a second end portion, and a fastener for removably fastening the first end portion to the second end portion. In some embodiments, the headband comprises a continuous loop (similarly to a rubber band), and the headband is elastic enough to be placed on the head of the wearer and taken off the head of the wearer without a releasable fastener. In some aspects, the headband can be configured to be stretched to have a loop length that is at least 110%, at least 120%, at least 130%, at least 140%, or at least 150% or more of the loop length in a non-stretched configuration. In some aspects, the headband can be stretched and released to its non-stretched configuration repeatedly without substantial loss of elasticity. In some embodiments, the headband is pivotable at the first strap coupling portion. For example, the first strap coupling portion can comprise a thinner or shorter portion, or comprise a different type of material, and an inner surface of the headband can comprise gripping material to help maintain a position of the headband on the wearer's head, for example, when one portion of the headband is pivoted relative to another portion. However, any suitable mechanism for pivoting one portion of a headband relative to another portion of the headband is contemplated. In some embodiments, the first strap comprises a looped portion sized and dimensioned to receive at least a portion of the first strap coupling portion. In some embodiments, at least one of the first strap and the second strap comprises a set of openings for ventilation comfort. In some embodiments, at least one of the first connector and the second connector comprises a set of openings for ventilation comfort. In some embodiments, at least one of the first strap and the second strap comprises a cooling portion (e.g., of a cooling gel material). In some embodiments, at least one of the first connector and the second connector comprises a cooling portion (e.g., of a cooling gel material). In some embodiments, the nasal cannula comprises a tubular base having an inlet for receiving the flow of fluid, a first prong and a second prong, wherein each of the first prong and the second prong is fluidly connected to the inlet, and wherein the first prong comprises a flexible portion comprising a set of ridges and grooves (e.g., at least 1 ridge and at least 1 groove, at least 2 ridges and at least 1 groove, at least 3 ridges and at least 2 grooves). Viewed from another perspective, a cannula comprising one or more bellows segments is provided. In some embodiments, the bellow segments can be provided on the nasal prongs, for example, on a lower portion of the nasal prong that is not inserted (or is only partially inserted) into a wearer's nostrils. The bellow prongs can advantageously have more degree of flexibility without collapsing or reducing the opening. The bellow segments can comprise a set of ring-shaped ridges and grooves, wherein the grooves can be entirely or partially collapsible such that a distance between the adjacent ridges can be adjustable along any portion of the bellow segments (e.g., one side of a ring-shaped groove collapsed by between 0.1 mm to 1 cm while another side of the same ring-shaped groove maintains its shape or expands to allow for an adjustment of an angle of the prong relative to the tubular base). It should be appreciated that any of the cannulas described herein can comprise one or more bellows segments, for example, bellow prongs and a bellow tubular base.

In another aspect of the disclosure, a patient interface assembly for a head of a wearer is provided, comprising a nasal cannula (or a mask such as an oxygen face mask, a nasal pillow, etc.), an elastic headband configured to wrap around at least a portion of the head of the wearer, a first connector extending from a first side of the nasal cannula (or a mask such as an oxygen face mask, a nasal pillow, etc.), a second connector extending from a second side of the nasal cannula (or a mask such as an oxygen face mask, a nasal pillow, etc.), and wherein the first connector and the second connector secure (e.g., removably secure, directly or indirectly) the nasal cannula (or a mask such as an oxygen face mask, a nasal pillow, etc.) to the elastic headband. In some embodiments, each of the connectors are configured to removably attach to the elastic headband at one or more locations via one or more fasteners (e.g., hook and loop fastener, snaps).

In some embodiments, the headband comprises a first end portion and a second end portion, and a fastener for removably fastening the first end portion to the second end portion. In some embodiments, the headband comprises a continuous loop (similarly to a rubber band), and the headband is elastic enough to be placed on the head of the wearer and taken off the head of the wearer without a releasable fastener. In some aspects, the headband can be configured to be stretched to have a loop length that is at least 110%, at least 120%, at least 130%, at least 140%, or at least 150% or more of the loop length in a non-stretched configuration. In some aspects, the headband can be stretched and released to its non-stretched configuration repeatedly without substantial loss of elasticity. In some embodiments, at least one of the first connector and the second connector is made at least in part of a silicone. In some embodiments, at least one of the first connector and the second connector comprises at least of a set of openings for ventilation comfort, and a cooling portion comprising a gel material. In some embodiments, each of the first connector and the second connector is made at least in part of a silicone. In some embodiments, each of the first connector and the second connector comprises at least of a set of openings for ventilation comfort, and a cooling portion comprising a gel material. In some embodiments, the first connector comprises a first end portion for removably coupling to a first portion of a headband via a first fastener. In some embodiments, the second connector comprises a second end portion for removably coupling to a second portion of a headband via a second fastener. In some embodiments, the first connector comprises a first end portion for removably coupling to a first strap via a first fastener, wherein the first strap is coupled to a first portion of a headband. In some embodiments, the second connector comprises a second end portion for removably coupling to a second strap via a second fastener, wherein the second strap is coupled to a second portion of a headband. In some embodiments, the nasal cannula comprises a tubular base having an inlet for receiving the flow of fluid, a first prong and a second prong, wherein each of the first prong and the second prong is fluidly connected to the inlet, and wherein the first prong comprises a flexible portion or bellow segment comprising a set of ridges and grooves.

In another aspect of the disclosure, a nasal cannula configured to deliver a flow of a fluid to a user is provided, the nasal cannula comprising a tubular base having an inlet for receiving the flow of fluid, a first prong and a second prong, wherein each of the first prong and the second prong is fluidly connected to the inlet, and wherein the first prong comprises a set of grooves.

In some embodiments, the first prong comprises a set of ridges. In some embodiments, the ridges of the set of ridges alternate with the grooves of the set of grooves. In some embodiments, the second prong comprises a second set of grooves. In some embodiments, the tubular base comprises a portion between the first and second prongs comprising a set of grooves. In some embodiments, the tubular base comprises a cooling portion made at least in part of a cooling gel material.

In another aspect of the disclosure, a headband configured to wrap around at least a portion of a head of a wearer is provided, comprising, an inner side and an outer side, and at least a first strap coupling portion. In some embodiments, the headband can further comprise a second strap coupling portion. In some embodiments, the first strap coupling portion comprises a shortened portion of the headband sized and dimensioned to couple with a first strap such that the first strap can slide along a length of the first strap coupling portion and be blocked by adjacent taller portions of the headband. In some embodiments, the second strap coupling portion comprises a shortened portion of the headband sized and dimensioned to couple with a second strap such that the second strap can slide along a length of the second strap coupling portion and be blocked by adjacent taller portions of the headband. In some embodiments, the headband comprises a first end portion and a second end portion, and a fastener for removably fastening the first end portion to the second end portion. In some embodiments, the headband comprises a continuous loop (similarly to a rubber band), and the headband is elastic enough to be placed on the head of the wearer and taken off the head of the wearer without a releasable fastener. In some aspects, the headband can be configured to be stretched to have a loop length that is at least 110%, at least 120%, at least 130%, at least 140%, or at least 150% or more of the loop length in a non-stretched configuration. In some aspects, the headband can be stretched and released to its non-stretched configuration repeatedly without substantial loss of elasticity.

The headbands described herein can be made of any suitable material or materials (e.g., synthetic material, natural material), can comprise any suitable number of layers (e.g., 1, 2, 3, 4, 5) or pieces, and can be elastic. In some embodiments, the headband can comprise a single or multi-layer synthetic material (e.g., neoprene). In some embodiments, the headband can comprise any suitable rubberized fabric, textile or material. In some embodiments, the rubberized material can be water resistant or waterproof. In some aspects, the rubberized material can provide a non-slip or grip or friction capability to the substrate material. In some embodiments, the headband can comprise multiple sections of materials of different elasticity. For example, a portion of the headband can be inelastic or less elastic than a second portion having greater elasticity.

The headbands described herein can comprise a strip of material comprising a first end portion with a first fastener component (e.g., hook or loop material), a second end portion with a second fastener component complementary to the first (e.g., loop or hook material), and can be configured to releasably fasten to one another to form a loop or band. The headband can comprise a multiple band portion (e.g., at a front, at a back of the wearer's head) wherein a first band portion is positioned over a second band portion. For example, a headband can comprise a front portion having a single band portion, and a back portion having two band portions (one band portion configured to be positioned around a top or crown region of a wearer's head, another band portion configured to be positioned around an occipital or nape region of a wearer's head). In some embodiments, the headband can comprise two or more portions wherein one portion is configured to be rotatable (e.g., pivotable up to 180 degrees) relative to another portion. In some embodiments, the headband can comprise three or more portions wherein a first portion is configured to be rotatable (e.g., pivotable up to 180 degrees) relative to a second portion, and a third portion is configured to be rotatable relative to the second portion.

A headband system can be provided, including a headband (e.g., any of the headbands described herein), and at least one strap. Each strap can be configured to adjustably couple to different portions of a strap coupling portion. For example, a first strap can be adjustably coupled to the first strap coupling portion, and a second strap can be adjustably coupled to the second strap coupling portion. The straps can couple to the strap coupling portions via any suitable mechanism. For example, a first end portion of a strap can include a mechanical fastener (e.g., hook and loop, snaps) configured to fasten to a complementary fastener of a strap coupling portion. As another example, the first end portion of the strap can comprise a looped portion (e.g., a sleeve, a loop formed between two slits) configured to receive at least a portion of a strap coupling portion. Each strap can comprise a second end portion opposite the first end portion, which can include a fastener for removably coupling the strap to a cannula connector (e.g., a cannula tab extending from a cannula), a cannula or other component. In some embodiments, the straps can comprise a set of openings for ventilation comfort/breathability. In some embodiments, the straps can comprise a cooling portion (e.g., a cooling gel material portion). In some embodiments, the straps can be rotatably coupled to the first strap coupling portion such that an angle formed by the headband and the strap can be adjusted. An inner surfaced of the straps can comprise a rubberized material or gripping material to help maintain a position of the strap relative to a wearer's head.

According to another aspect, a cannula (or other interface) connector for an air flow system cannula (or other interface) to be worn by a wearer is provided, comprising a tab having any suitable length and made of any suitable material that extends from or is configured to couple the cannula, wherein the tab has a convex shape such that edge portions of the at least one of the tab and the strap face away from the cheek of the wearer when worn. In some embodiments, the tab has a thickness of between 0.1-4 mm. in some embodiments, the cannula connector consists of a single piece of material having a first end that couples to or extends from the cannula and a second end that couples to a headband. In some embodiments, the second end comprises a loop sized and dimensioned to receive an end portion of a strap coupled to the headband such that the end portion of the strap can be looped through the loop and fasten to itself (e.g., via hook and loop fastener). In some embodiments, the cannula connector is made partially or entirely of a silicone material and is elastic. However, all suitable materials are contemplated. In some embodiments, the cannula connector comprises a double tensioning mechanism from elasticity and an adjustable fastener. In some embodiments, the strap is adjustably coupled to each of the tab and the headband. In some embodiments, at least one of the tab and the strap comprises ventilation openings. In some embodiments, at least one of the tab and the strap is angled to contour to the wearer's face. In some aspects, at least one of the tab and the strap can comprise multiple sections of materials of different elasticity. For example, a portion of the tab and/or strap can be inelastic or less elastic than a second portion having greater elasticity.

According to another aspect, a cannula apparatus is provided, comprising: a cannula (e.g., a nasal cannula) or other interface (e.g., mask); and a strap configured to wrap around a back of a wearer's head, and to couple to the cannula or other interface. In some embodiments, the strap can comprise a single piece/band. In some embodiments, the strap can comprise two or more pieces/bands that couple to one another. In some embodiments, the strap is configured to indirectly couple to the cannula, for example, via one or more cannula tabs. In some embodiments, the strap comprises a single band. The strap can be made of any suitable material or materials (e.g., synthetic material, natural material), can comprise any suitable number of layers (e.g., 1, 2, 3, 4, 5) and pieces, and can be elastic. In some aspects, the strap can comprise a single or multi-layer synthetic material (e.g., neoprene). In some aspects, the strap can comprise any suitable rubberized fabric, textile or material. In some aspects, the rubberized material can be water resistant or waterproof. In some aspects, the rubberized material can provide a non-slip or grip or friction capability to the substrate material. In some aspects, the strap can comprise multiple sections of materials of different elasticity. For example, a portion of the strap can be inelastic or less elastic than a second portion having greater elasticity.

In some embodiments, the strap can comprise one or more fasteners. In some embodiments, the strap comprises a first piece and a second piece, wherein the first piece has a first end portion and a second end portion, wherein the second piece has a third end portion and a fourth end portion, wherein the first end portion is configured to couple to at least one of the cannula or other interface and a first tab (e.g., a first tab extending from a first side of the cannula), wherein the fourth end portion is configured to couple to at least one of the cannula or other interface and a second tab (e.g., a second tab extending from a second side of the cannula opposite the first side), and wherein the second end portion and the third end portion are configured to removably fasten to one another via a first fastener. In some embodiments, the strap comprises a single piece having a first end portion and a second portion, wherein the first end portion is configured to couple to at least one of the cannula or other interface and a first tab at least one of coupled to or extending from the cannula or other interface, and wherein the second end portion is configured to couple to at least one of the cannula or other interface and a second tab at least one of coupled to or extending from the cannula or other interface.

According to another aspect, a tab is provided comprising at least one of extending from a side of a cannula or configured to couple with a cannula (e.g., a nasal cannula), is provided, and configured to adhere to at least one of a wearer's skin and a frame worn by the wearer. In some embodiments, the cannula tab is configured to adhere to (or otherwise couple to) the at least one of the wearer's skin and the frame worn by the wearer via an adhesive material and/or fastener (e.g., for removable fastening) and/or negative pressure. In some embodiments, the cannula tab is configured to adhere to the at least one of the wearer's skin and the frame worn by the wearer via negative pressure. In some embodiments, the frame comprises a nose frame. In some embodiments, the frame comprises a nose frame, and further comprising a fastener configured to adhere the cannula tab to the nose frame. In some embodiments, the fastener comprises a hook and loop fastener.

In some embodiments, any of the cannulas, the headbands, the straps, the cannula connectors (e.g., tabs) or any other component described herein can comprise cooling portions, for example cooling gel portions. The cooling portions can be positioned on surfaces of the cannula, headband, strap, tab, etc. configured to contact a wearer's skin. The cooling gel or other cooling material can be provided in any suitable manner using any suitable materials. In some embodiments, the cannula tabs can be constructed using open cell foam or they can be inserted with gel beads. In some embodiments, the cooling gels can be trapped within a top layer and adhere to the cannula tabs. In some embodiments, the cooling gels can comprise any suitable size and shape. The cooling gels/materials can advantageously draw heat away from the body and release it quicker.

Other advantages and benefits of the disclosed assemblies, components and methods will be apparent to one of ordinary skill with a review of the following detailed description.

### BRIEF DESCRIPTION OF THE DRAWINGS

The details of embodiments of the present disclosure, both as to their structure and operation, may be gleaned in part by study of the accompanying drawings, in which like reference numerals refer to like parts, and in which:

FIG. 1A illustrates a headband, a cannula, cannula connectors, and straps of an air flow system, according to an embodiment;

FIGS. 1B-1D illustrates components of an air flow system positioned on a model of a head;

FIG. 2A illustrates a first side of a headband, according to an embodiment;

FIG. 2B illustrates a first side of the headband of FIG. 2A with a position of the straps adjusted;

FIG. 2C illustrates a second side of the headband of FIG. 2B;

FIG. 2D illustrates a first side of a strap and strap coupling portion of FIG. 2A;

FIG. 2E illustrates a second side of the strap and strap coupling portion of FIG. 2D;

FIG. 2F illustrates a top view of the headband of FIG. 2A showing three layers with rubber in the mid-layer for elasticity and retention;

FIG. 3 illustrates a cannula and cannula connectors (e.g., wing tabs), according to an embodiment;

FIG. 4 illustrates a cannula and cannula connectors (e.g., wing tabs) with vent holes, according to an embodiment;

FIG. 5 illustrates a cannula with cannula connectors (e.g., wing tabs) that removably fasten to strap coupling portions (or any other portion) of a headband;

FIGS. 6A-6C illustrates a cannula with cannula connectors removably fastened to a headband and worn on a model of a wearer's head, according to an embodiment;

FIG. 7A illustrates a cannula with cannula tabs coupled to a nose frame, according to an embodiment;

FIG. 7B illustrates the cannula with cannula tabs coupled to a nose frame of FIG. 7A worn on a wearer's face;

FIG. 8A illustrates another cannula with cannula tabs, according to an embodiment;

FIG. 8B illustrates the cannula with cannula tabs of FIG. 8A adhered to a wearer's face;

FIG. 9A illustrates a cannula with flexible bellow prongs, according to an embodiment;

FIG. 9B illustrates the cannula with flexible bellow prongs of FIG. 9A worn on a wearer's face; and

FIG. 10 illustrates a cannula device comprising cooling gel portions on an inner surface configured to contact a wearer's skin, according to an embodiment.

### DETAILED DESCRIPTION

The detailed description set forth below, in connection with the accompanying drawings, is intended as a description of various embodiments, and is not intended to represent the only embodiments in which the disclosure may be practiced. The detailed description includes specific details for the purpose of providing a thorough understanding of the embodiments. However, it will be apparent to those skilled in the art that embodiments of the invention can be practiced without these specific details. In some instances, well-known structures and components are shown in simplified form for brevity of description.

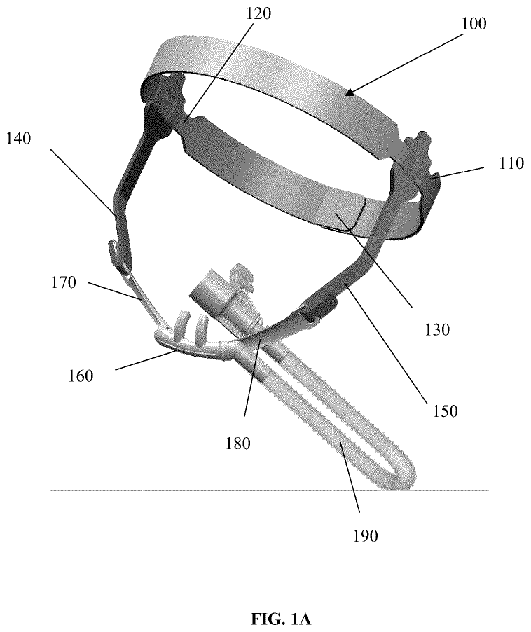

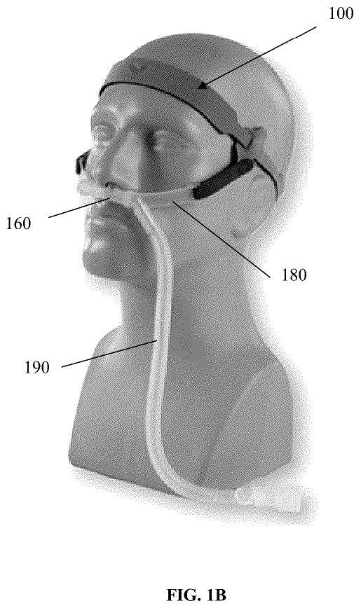

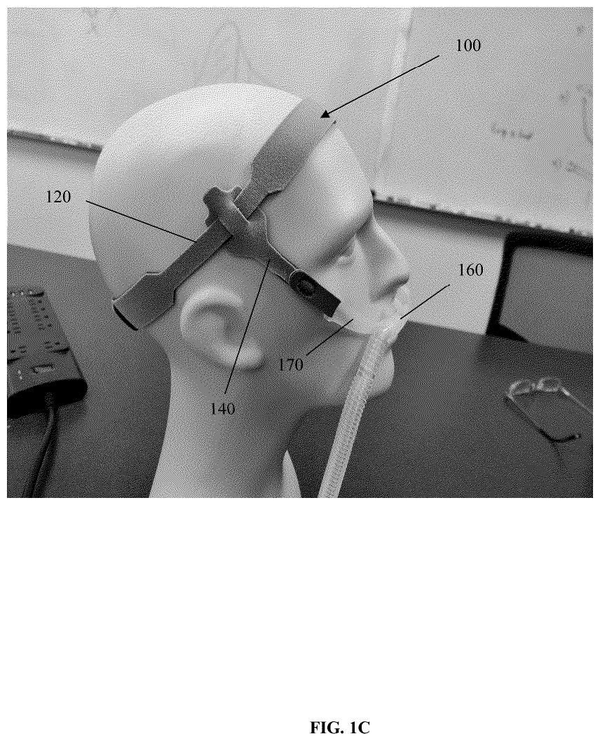

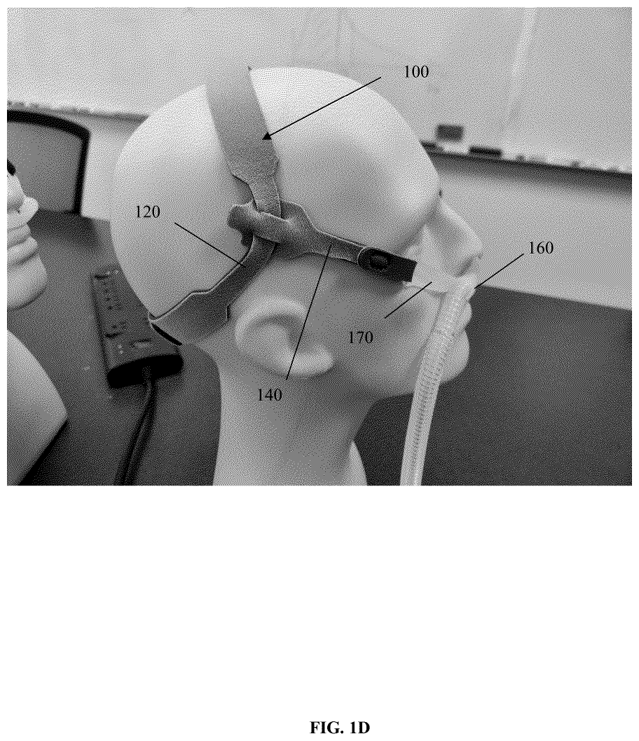

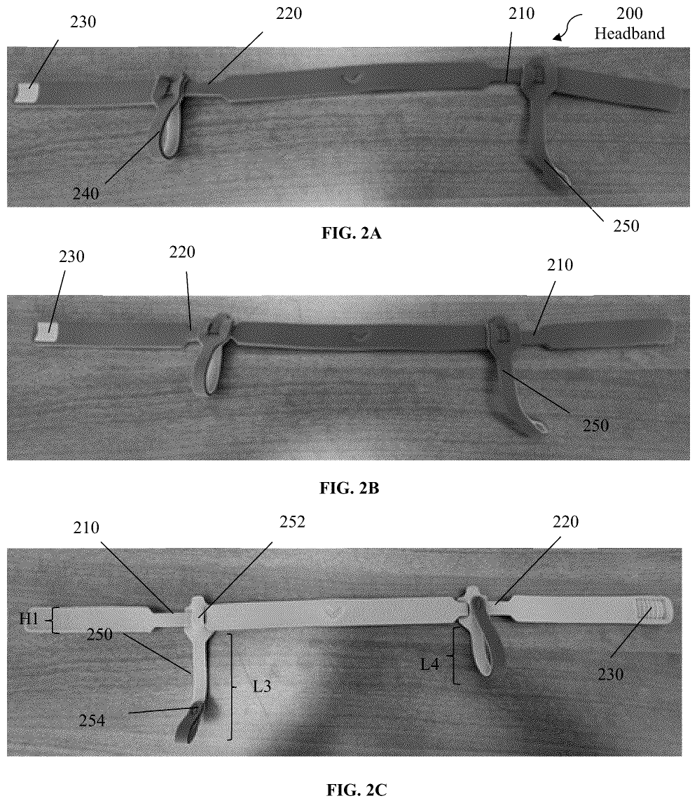

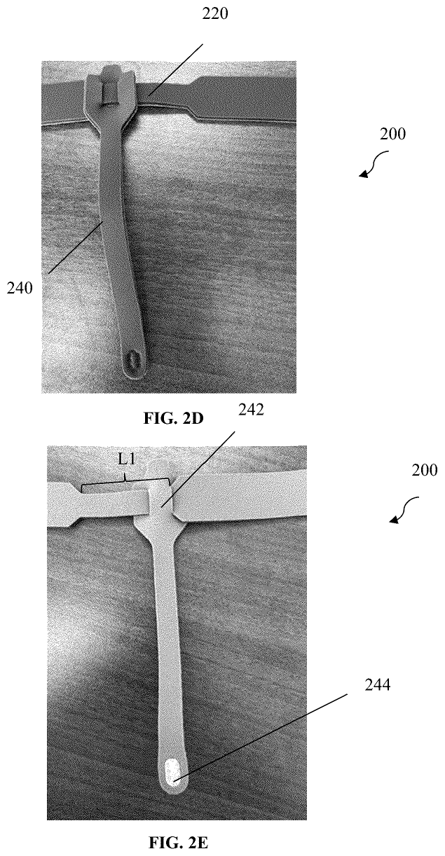

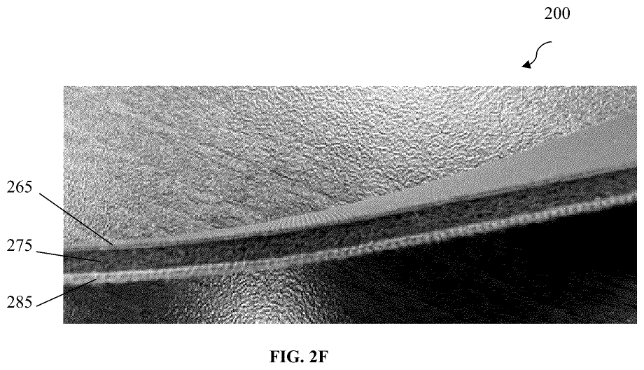

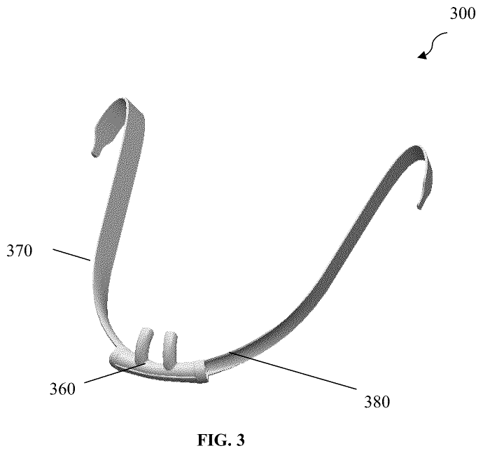

Headbands, cannula connectors, straps, nose frames, and other components of air flow systems are described herein. As illustrated in FIG. 1A, components of an air flow system can comprise a headband 100, a cannula 160, tubing 190, first and second cannula connectors 170, 180 extending from opposite sides of cannula 160, and first and second straps 140, 150, each of which is adjustably coupled to a cannula connector and a strap coupling portion 110, 120 of headband 100. Headband 100 can have some or all of the same features of headband 200, and can comprise a continuous loop, or can comprise a first end portion and a second end portion configured to releasably and/or adjustably fasten to one another at fastened end portions via a fastener 130 (e.g., hook and loop, snaps). FIGS. 1B-1D illustrates components of FIG. 1A as worn on models of a human head. In FIG. 1B, the strap is shortened by adjusting a position of where an end portion fastener component (e.g., hook material on the strap) is fastened to a complementary fastener component (e.g., an outer loop material of the strap, or a loop material coupled to the strap). In FIG. 1C, the strap is longer than as shown in FIG. 1B. This adjustment can be based on, among other things, differences in head sizes and shapes, differences in facial feature shapes, sizes and locations, and/or based on whether the cannula should fit loosely or snugly. The strap length (as worn) can be adjustable by any suitable amount (e.g., difference between L3 and L4 in FIGS. 2C, e.g., between 1-15 cm, between 1-10 cm, between 1-8 cm, between 1-5 cm, between 0.1-5 inches, between 0.1-3 inches, between 0.1 to 1 inch). In FIG. 1D, the headband has been adjusted on the wearer so that a front portion (e.g., portion in front of the strap coupling portions) is pivoted or otherwise rotated to be higher on the wearer's head relative to the front portion in FIGS. 1B-1C. It is contemplated that the headband can rotate (e.g., pivot), for example at the strap coupling portions, such that the front portion rotates relative to the back portion (e.g., portion behind the strap coupling portions), upwards or downwards, by any suitable amount (e.g., up to 180 degrees, up to 135 degrees, up to 90 degrees). A surface of the headband that contacts that wearer's head can include a gripping material, such as rubber or silicone to help maintain a position of a portion of the headband relative to another portion of the headband, or relative to a portion of a wearer's head (e.g., a top of a forehead). All suitable cannulas, tubing, cannula tabs or other connectors, and other components are contemplated for use with the headbands and/or straps described herein, including the cannulas and tubing described in International Patent Application No. PCT/US2022/028737, filed on May 11, 2022, which is incorporated by reference herein in its entirety.

FIGS. 2A-2F illustrate a headband with two straps 240, 250 coupled to strap coupling portions 210, 220 of a headband 200. A first side, which is described herein as the side facing the wearer's head, is shown in FIGS. 2A-2B, and 2D. A second side, which is described herein as the side facing away from the wearer, is shown in FIGS. 2C and 2E. FIG. 2F shows a top view of the headband where a thickness of the headband can best be seen. In the example shown, the headband is made from a three-layer neoprene material having a first surface layer 265, a second surface layer 285, with a rubber mid-layer 275 for elasticity and retention. However, it should be appreciated that a headband and other components described herein can be made of any suitable material(s) having one or several layers.

It should be appreciated that the first side and the second side can be switched in some embodiments such that the first side faces away from the wearer and the second side faces the wearer's head.

In the embodiment shown in FIGS. 2A-2F, headband 200 comprises a strip of material that is preferably somewhat elastic (e.g., neoprene, rubber, rubberized material, or a combination thereof) having a first end portion and a second end portion. One end portion comprises a band fastener component (e.g., hook or loop) 230 that is configured to removably fasten to a complementary fastener component of the other end portion (e.g., on second side). The headband can have any suitable length from one end (e.g., adjacent where fastener component 230 is positioned) to the other end of headband, including for example, between 10-20 inches, between 8-18 inches, between 8-16 inches, or any other suitable length. The complementary fastener component can be the outer material of the second side of the strip of elastic material, or can be coupled to a side (e.g., inner side, outer side) of the strip of elastic material (or any other suitable portion of the strip of elastic material), for example, a hook or loop material. The complementary fastener can be larger than the band fastener component 230, and/or there can be multiple complementary fasteners at different positions of the strip of material such that the first and second end portions can adjustably fasten to one another to form bands of different sizes and diameters. Exemplary fasteners include hook and loop fasteners or button or ladder lock fasteners. However, all suitable fasteners are contemplated herein. While headband 200 comprises a single strip of material that is configured to wrap around a head of a wearer, it should be appreciated that the headband can comprise multiple pieces of material, for example, multiple strips that are adjustably and/or rotatably (e.g., pivotably) coupled to one another.

Headband 200 comprises a strap coupling portion 210 and strap coupling portion 220. Each strap coupling portion acts as an anchor point to a strap (e.g., 240, 250). The straps can be made of any suitable material or materials (e.g., neoprene, silicone, rubber, rubberized fabric), and can be elastic. The strap coupling portions can comprise shorter portions of the headband between taller portions, with the taller portions acting as blocks or stops beyond which a strap may be prevented from moving. Any suitable length L1 is contemplated for the strap coupling portions, for example, between 0.5-3 inches, between 0.5-2 inches, between 1-3 inches, between 0.25-1.75 inches, or any other suitable length. The strap can have a first end portion and a second end portion, wherein the first end portion is configured to anchor to the headband and comprise a looped portion (e.g., between slit widths, a pocket, a sleeve). The first end portion can be configured to slide along a length of a strap coupling portion while being blocked by the taller portions. Such an arrangement allows for a controlled adjustment of a position of a strap 240, 250 relative to headband 200. One or more of an inner surface of a loop or a surface of headband 200 can comprise a gripping material (e.g., a rubber). As an example, FIG. 2A shows straps 240, 250 further apart from one another (e.g., at least 1 cm, at least 2 cm, at least 3 cm, at least 4 cm, at least 5 cm further apart) than in FIG. 2B via an adjustment of the straps relative to the strap coupling portions. It should be appreciated that other mechanisms that allow for straps 240, 250 to be adjustably coupled to headband 200 are contemplated herein. It should also be appreciated that the straps can couple to strap coupling portions in any suitable manner, including snaps, looping an end of a strap over strap coupling portion and fastening the end of the strap to another portion of the strap (e.g., can have a headband anchoring end that is similar to the other end of the strap that can include fastener component 254), hook and loop fasteners, etc. In some aspects, the straps can be configured to rotate (e.g., pivot) relative to headband. In some aspects, a strap can be configured to slide along a strap coupling portion's length and be positioned in two or more positions that are at least 1 cm, at least 2 cm, at least 3 cm, at least 4 cm, or at least 5 cm apart. Viewed from another perspective, a midpoint of a looped portion at a first position and a midpoint of a looped portion at a second position can be at least 1 cm, at least 2 cm, at least 3 cm, at least 4 cm, or at least 5 cm apart.

The second end of a strap can comprise a strap fastener component 244, 254 (e.g., a hook or loop material), which can be configured to fasten second end portion to another portion of the strap having a complementary fastener component. The complementary fastener component(s) can be the outer surface material of the strap, or can be coupled to the strap. In some aspects, the fastener of the strap can comprise a hook and loop fastener, a button, a ladder lock, or any other suitable fastener(s). In the embodiment shown in FIGS. 2A-2F, the complementary fastener component is an outer material of the strap, and strap fastener component 244, 254 can be removably coupled to the outer material of the strap at various positions. See, for example, the straps in FIG. 2C. As best seen in FIG. 1B-1D, the second end of the strap can thread through a slot in a cannula tab and fasten to the strap at various positions. The first end of the strap can be adjusted along a strap coupling portion of the headband. These features advantageously allow the strap to be adjusted in multiple ways based on the size and shape of the wearer's head.

The headband can have any suitable height/heights H1, including for example, 0.5-1 inch, 0.1-2 inches, or any other suitable height. It is contemplated that a taller headband can provide more comfort/less pressure to a wearer's head. In some aspects, the taller portion has a height that is at least 10%, at least 25%, at least 35%, or at least 50% greater than a height of the shorter strap coupling portions. In some aspects, the taller portion has a height of between 0.2-1.3 inches, between 0.5-1.3 inches (or any other suitable height), and the shorter portion has a height of between 0.1-1 inch, between 0.2-0.8 inches (or any other suitable height).

In some contemplated embodiments, a strap can couple to a single piece cannula (e.g., direct or indirect coupling). In some contemplated embodiments, a cannula connector can extend from or couple to a cannula, and can couple to the headband (e.g., direct or indirect coupling). In some contemplated embodiments, a cannula can be coupled to a headband via one or more tabs and one or more straps. In some embodiments, a cannula assembly lacks a tab. In some embodiments, a cannula assembly lacks a strap.

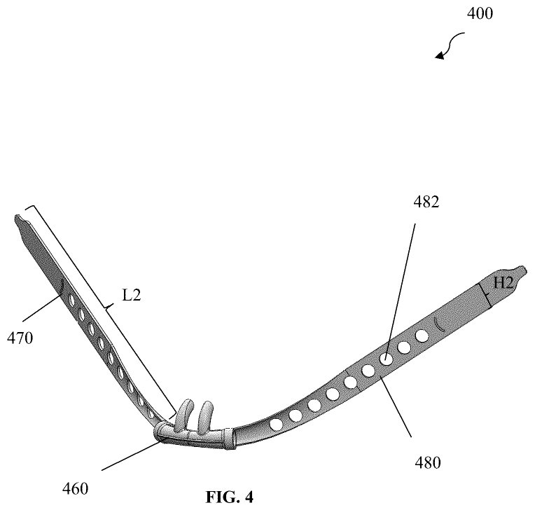

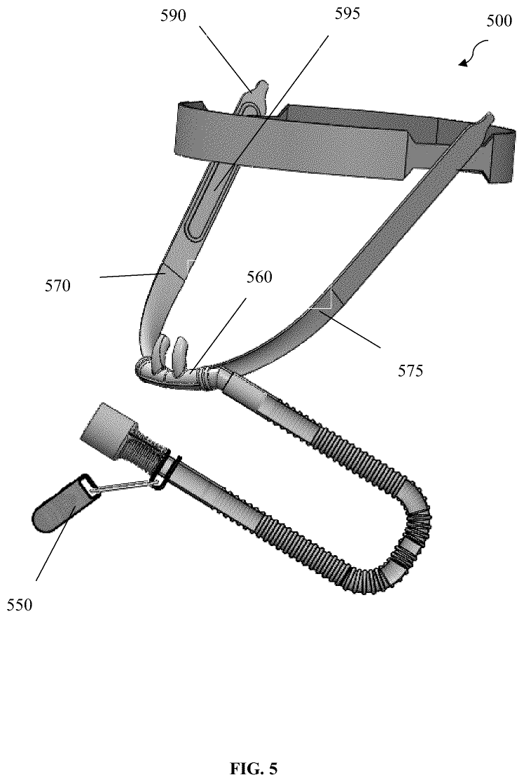

FIG. 3 illustrates a patient interface assembly 300 comprising a cannula 360 and cannula connectors 370, 380 (here, long wing tabs), according to an embodiment. Each cannula wing tab can be any suitable size and shape (e.g., each connector can have a length of between 0.5-12 inches, between 0.5-8 inches, between 0.5-5 inches, between 0.5-3 inches, between 0.5-2 inches, or any other suitable length), and can be made of any suitable material. In some embodiments, the cannula wing tabs can be made of the same material as cannula 360. In some embodiments, the cannula wing tabs and cannula can be formed of a single piece of material. FIG. 4 illustrates another patient interface assembly 400 comprising cannula 460 and cannula connectors 470, 480, according to another embodiment. The connectors of the embodiment of FIG. 4 differ from those in FIG. 3 in that they include openings 482 for ventilation comfort/breathability.

In some embodiments, the cannula and cannula connectors can comprise a single piece. In some embodiments, the cannula connectors 370, 380 can be removably or non-removably (directly or indirectly) coupled to opposite sides of cannula 360. In some embodiments, the cannula connectors can each have a free end configured to couple to at least one of a strap and a headband (or other headgear). For example, the cannula connectors can couple directly to a strap and/or a headband.

It is contemplated that the cannula connectors and straps can be made of any suitable material(s), including, for example, a silicone, a soft plastic, a neoprene, a rubberized material, a rubber, or any other suitable material(s). In some aspects, the connectors and straps can comprise the same materials. In some aspects, the connectors and straps can be made of different materials. In some aspects, the connectors can be angled to contour to a wearer's face. In some aspects, the cannula connectors can be convex with edges and/or corners facing away from a patient's face. In some aspects, the connectors can have a convex radius/angle that ranges from 0.1-180 degrees. In some aspects, a cannula connector can have a thickness of 0.1-10 mm, 0.1-4 mm, although any suitable thickness(es) are contemplated herein. In some aspects, a cannula connector can have a height H2 of between 0.1-1.5 inches, between 0.1-1 inch, or any other suitable height. In some aspects, a cannula connector can have any suitable length L2, for example, based on whether a strap or other connectors will be used (e.g., between 1-15 inches, between 1-12 inches, between 1-10 inches, between 1-8 inches, between 1-6 inches, between 1-5 inches), and the length can be adjustable. In some aspects, the straps can be angled to contour to a wearer's face. In some aspects, the straps can be convex with edges and/or corners facing away from a patient's face. In some aspects, the straps can have a convex radius/angle that ranges from 0.1-180 degrees. In some aspects, the straps can have a thickness of 0.1-10 mm, or 0.1-4 mm, although any suitable thickness(es) are contemplated herein. In some aspects, a strap can have a height of between 0.1-1.5 inches, between 0.1-1 inch, or any other suitable height. In some aspects, a strap can have any suitable effective length when coupled to the headband and a connector or cannula (as seen in FIGS. 2A-2B without cannula or connector, L3 and L4) (e.g., between 1-15 inches, between 1-12 inches, between 1-10 inches, between 1-8 inches, between 1-6 inches, between 1-5 inches), and the length can be adjustable.

The cannula connectors or straps can couple directly or indirectly to a headband or other device via any suitable fastening mechanisms (e.g., hook or loop on an end portion of the connector or strap with complementary hook or loop on surface material of a headband, button and complementary hole, snaps, loop around or an end portion and fastening to an opposite side of the connector or strap—e.g., via hook and loop). It is contemplated that the connectors or straps can attach or otherwise couple to the headband at multiple locations. A tensioning mechanism can be provided from elastic silicone tabs. While each cannula connectors is shown as a single piece of material, it should be appreciated that suitable connectors can be a single continuous piece of material or multiple materials linked together.

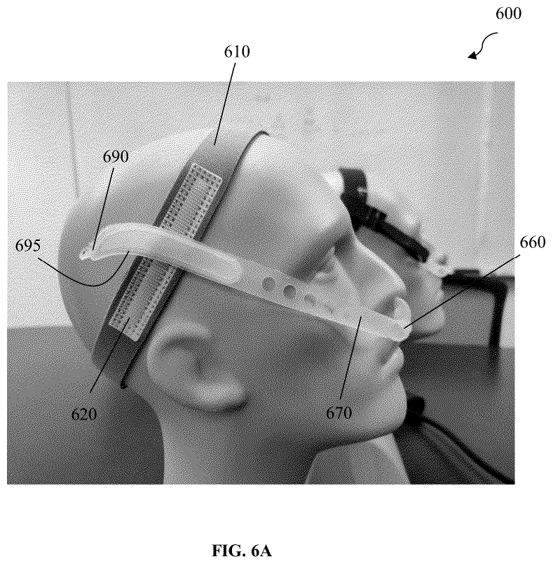

FIG. 5 illustrates a user interface assembly 500 comprising a cannula 560 with cannula connectors (e.g., wing tabs) 570, 575 that removably fasten to strap coupling portions (or any other portion) of a headband via fastener components (e.g., 595) on free end portions (e.g., 590) of the connectors. In some aspects, the fastener component can comprise a hook or loop material attached to the connector, and a complementary fastener can be provided on the headband. The complementary fastener can be, for example, the loop material outer layer of the headband (and/or a fastener material attached to the headband), and the fastener component of the cannula connector can be configured to removably fasten to any suitable portion of the headband (e.g., strap coupling portion, front portion, rear portion). All other suitable fasteners are contemplated herein, including buttons and buttonholes, snaps, etc. In some embodiments, a clip 550 can be provided (e.g., on tubing coupled to an inlet of cannula 560), which can be coupled to an item worn by the wearer (e.g., gown, shirt, collar). In some embodiments, the connector (e.g., a free end portion of the connector) can be at least one of pre-cut, serrated, and perforated such that one or more pieces can be torn off to reduce the amount of the connector sticking out beyond the point where it is fastened to the headband. For example, a connector can comprise 0, at least 1, at least 2, or at least 3 or more cutaway portions that can be easily removed at the at least one of pre-cut, serrated, and perforated lines.

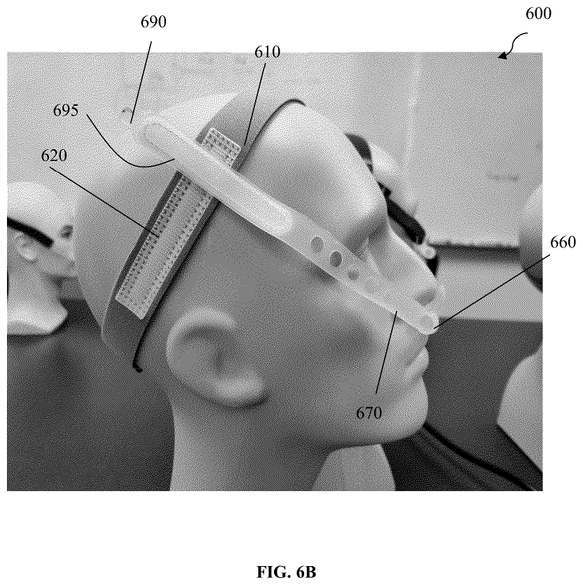

FIGS. 6A-6C illustrates an assembly 600 including a cannula 660 with cannula connectors (e.g., 670) removably fastened to a headband 610 and worn on a model of a wearer's head, according to an embodiment. The cannula connector 670 comprises a first fastener component (e.g., hook or loop) 695 at a first end portion 690, and the headband comprises a second fastener component 620, complementary to the first fastener component. The cannula connector is similar to the connectors shown in FIG. 5, but further includes vent holes. As shown in FIGS. 6A, 6B, and 6C, the location where the cannula connector fastens to the headband can be adjusted in various ways. In FIG. 6A, connector 670 is positioned about midway along fastener 620. In FIG. 6B, connector 670 is positioned near a first end of fastener 620. In FIG. 6C, connector 670 is positioned near a second end of fastener 620 opposite the first end. It is contemplated that the fastener 620 can comprise any suitable length to allow for repositioning of connector 670. For example, the fastener 620 can have a length of between 1-10 inches, between 1-5 inches, or between 2-5 inches.

In some aspects, the wing tabs or straps can be sewn on, adhered, or otherwise attached to a different materials and/or components, and such different materials and/or components can, for example, removably fasten to one another (e.g., at a back of a wearer's head), and/or removably fasten to/couple to another component (e.g., cannula, cannula connector component, headband).

In some aspects, a cannula with a single strap that is configured to loop around a head of a wearer is provided. The single strap can include two end portions, wherein a first end portion couples to a first side of a cannula and a second end portion couples to a second side of the cannula. The single strap can be made of an elastic material (e.g., neoprene, rubberized material, rubber) such that it can be stretched and placed on a wearer's head. The strap can comprise openings, for example, similar to openings 482 for ventilation comfort. The strap can be convex with edges and/or corners facing away from a patient's face. The strap can have any suitable thickness or thicknesses (e.g., 0.1-4 mm). The strap can have any suitable height (e.g., between 0.1-2 inches).

According to another aspect, a connector at least one of extending from and configured to couple to a cannula (e.g., a nasal cannula), and at least one of extending from and configured to couple to a frame (e.g., a nose frame) worn by the wearer (e.g., on the wearer's face) is provided. In some embodiments, the frame is made of any suitable material, for example, a metal, a plastic, a shape memory material, or any other suitable material. In some embodiments, the connector is configured to adhere to at least one of a wearer's skin and the frame. In some embodiments, the cannula connectors each form an angle of between 45-135 degrees with a tubular base of the cannula. In some embodiments, the cannula connectors each form an angle of between 45-135 degrees with the frame. In some embodiments, the cannula connectors are configured to adhere to the at least one of the wearer's skin and the frame worn by the wearer via an adhesive material. In some embodiments, the cannula connector is configured to adhere to the at least one of the wearer's skin and the frame worn by the wearer via negative pressure. In some embodiments, the frame comprises a nose frame. In some embodiments, the frame comprises a nose frame, and further comprising a fastener configured to adhere the cannula tab to the nose frame. In some embodiments, the fastener comprises a hook and loop fastener.

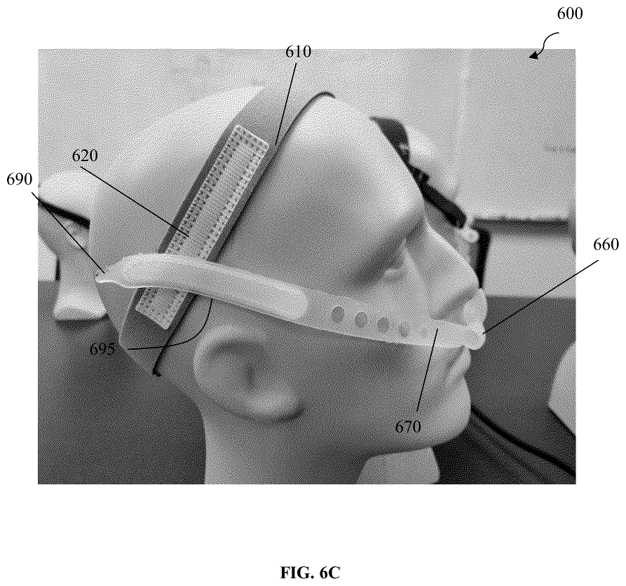

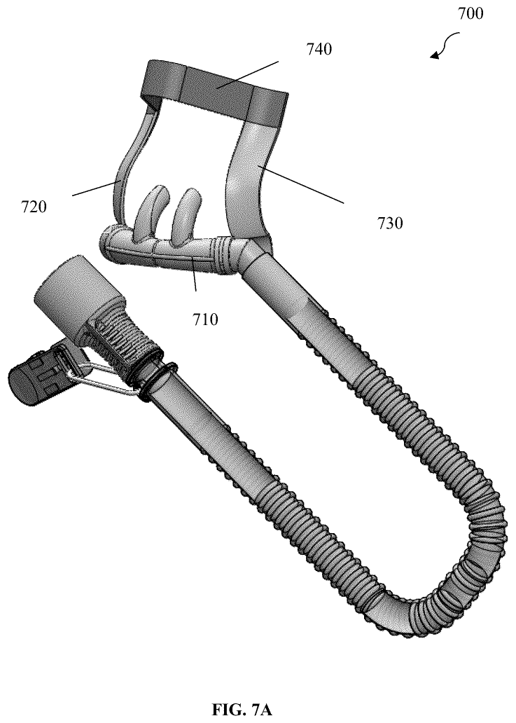

FIGS. 7A-7B illustrate an assembly 700 comprising a cannula 710 with cannula connectors (e.g., tabs) 720, 730 extending from opposite sides of cannula 710. The cannula 710 can be held in place on a wearer's face via a nose frame 740, which the connectors can be coupled to, or removably couple to. In some embodiments, the nose frame, cannula tab(s) and/or cannula can comprise an adhesive, such as a topical skin adhesive or any other suitable adhesive. The cannula connectors can comprise any suitable length (e.g., between cannula and frame when worn), for example, between 1-10 cm, between 1-8 cm, or between 1-5 cm. In some embodiments, a length of the cannula connectors between the cannula and frame can be adjustable (e.g., an end portion of the connectors can include a fastener configured to removably and adjustably attach to nose frame 740).

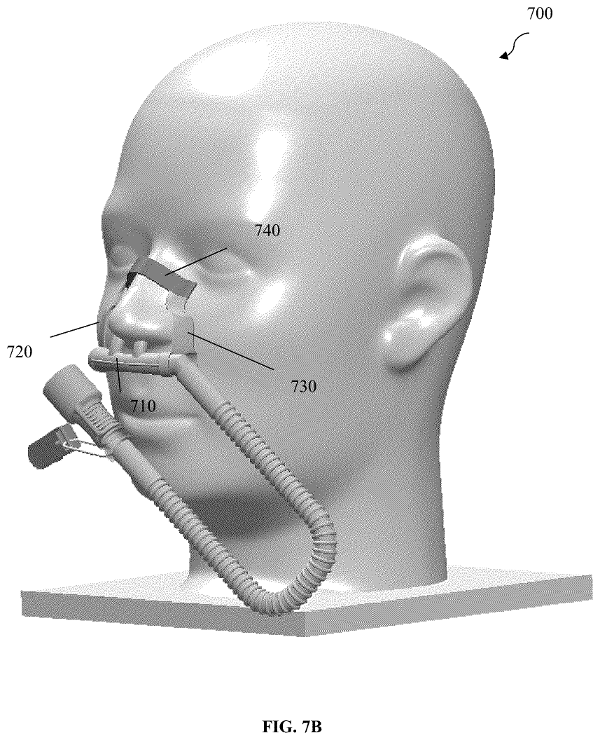

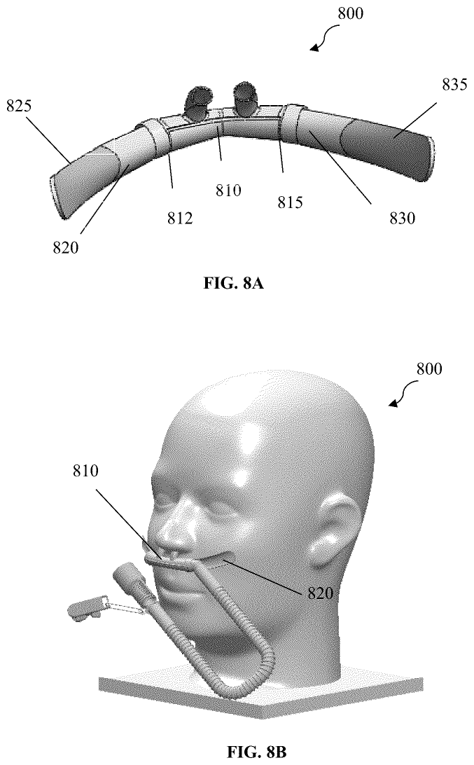

FIGS. 8A-8B illustrate an assembly 800 comprising a cannula 810 with cannula connectors 820, 830 configured to be adhered to a wearer's face, according to an embodiment. Cannula 810 has a first side portion 812 and a second side portion 815. Cannula connector 820 at least one of extends from and is coupled to a first end portion 812, and cannula connector 830 at least one of extends from and is coupled to a second end portion 830. Cannula connectors 820 and 830 comprise an adhering portion 825, 835 comprising an adhesive (e.g., a topical skin adhesive, an acrylic polymer, pressure sensitive acrylic adhesive) and configured to adhere to at least one of a wearer's skin and an object worn by the wearer (e.g., a nose frame or other frame). A backing can be provided over adhering portions 825, 835, which can be removed prior to use.

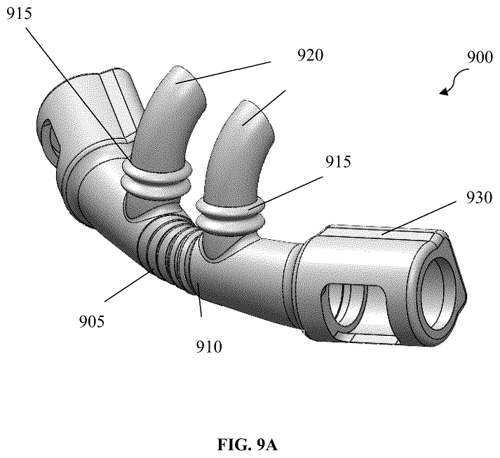

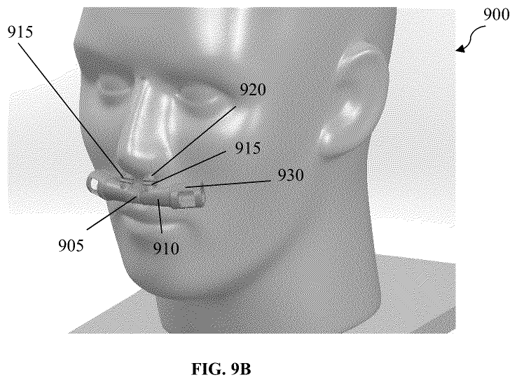

FIGS. 9A-9B illustrate a cannula 900 comprising a tubular base 910 including an inlet portion 930, with flexible or articulated prongs 920 extending from a mid-portion of tubular base at an angle, each having an angle adjustable bellow segment 915. The bellow segment 915 can comprise a concertina like hinge. The angle adjustable bellow segment 915 can comprise at least one ring-shaped groove (smaller diameter portion relative to adjacent portions), for example, between two or more ring-shaped ridges (larger diameter portions relative to an adjacent portion(s)). In some embodiments, the ring-shaped groove(s) can be smaller diameter portions of prongs 920. In some embodiments, the ring-shaped grooves can comprise a smaller, the same or greater diameter as the non-bellow segment portion of prongs 920, and can be positioned between ring-shaped ridges having a greater diameter. In some embodiments, the ridges and grooves can be rounded. In some embodiments, the ridges and grooves can be folded. In some embodiments, prongs 920 can be made of any suitable material, including a silicone, etc. In some embodiments, bellow segment 915 can be made of any suitable material, which can be the same as, or different from, the rest of the prong 920. In some embodiments, a bellow segment 915 can have the same or different thickness (i.e., wall thickness) as the rest of prong 920. Bellow prongs can advantageously have more degree of flexibility without collapsing or reducing the opening. In some embodiments, the bellows can contract and expanse without affecting the diameter of the prong openings, for example, due to the shape of the bellow and the additional material provided in that shape. In some embodiments, the bellows can allow for angle adjustment, or shortening or lengthening of the prongs, and can be configured to maintain various configurations (e.g., maintain a collapsed configuration, maintain an expanded configuration, maintain an angled configuration), for example, until manually adjusted. In some embodiments, one or more prongs 920 can comprise one or more bellows segments. In some embodiments, a bellows segment can be provided on a lower portion of bellow prongs 920 (e.g., near tubular base 910). In some embodiments, a portion of the cannula other than the prongs (e.g., tubular base 910) can comprise one or more bellows segments (e.g., 905). For example, a portion of the tubular base 910 positioned between bellow prongs 920 can comprise a bellows segment.

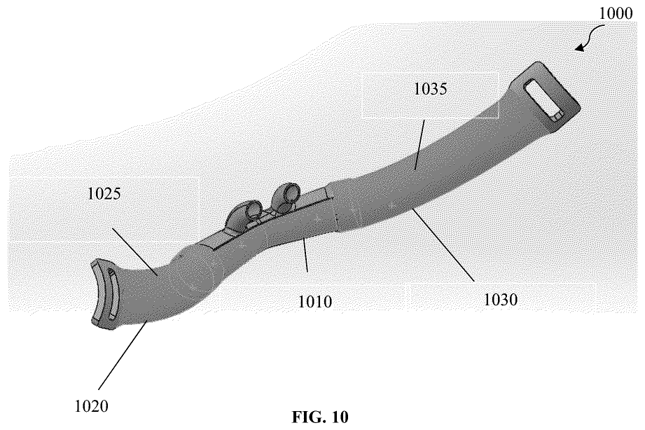

FIG. 10 illustrates a cannula device 1000 comprising a cannula 1010 with cannula connectors 1020, 1030 having cooling portions 1025, 1035 on surfaces configured to contact a wearer's skin. The cooling surfaces can each comprise a cooling gel (e.g., gel beads) or any other suitable cooling material provided in any suitable manner using any suitable materials. In some embodiments, the cannula connectors can be constructed using open cell foam or they can be inserted with gel beads. In some embodiments, the cooling gels can be trapped within a top or surface layer and adhere to the cannula connectors. In some embodiments, the cooling portions can comprise any suitable size and shape. In some embodiments, a cannula connector (e.g., a cannula wing tab) can comprise a single cooling gel portion or a set of cooling gel portions that are positioned together or separated from one another. In some embodiments, a strap (e.g., that couples a connector to a headband or other head gear) can comprise a single cooling gel portion or a set of cooling gel portions that are positioned together or separated from one another. In some embodiments, a cannula can comprise a single cooling gel portion or a set of cooling gel portions that are positioned together or separated from one another. The cooling gels/materials can advantageously draw heat away from the body and release it quicker.

According to another aspect, a cannula apparatus including a headband is provided, comprising: an air flow cannula; a first strap having a first end portion and a second end portion, the first end portion configured to couple to the air flow cannula, and the second end portion configured to couple to the headband; and a second strap having a third end portion and a fourth end portion, the third end portion configured to couple to the air flow cannula, and the fourth end portion configured to couple to the headband. In some embodiments, each of the first strap and the second strap is configured to indirectly couple to the air flow cannula, for example, via one or more cannula connectors. In some embodiments, the headband comprises: an inner side and an outer side; a first strap coupling portion; and a second strap coupling portion. In some embodiments, the first strap coupling portion comprises a shortened portion of the headband sized and dimensioned to couple with a first strap such that the first strap can slide along a length of the first strap coupling portion. In some embodiments, the second strap coupling portion comprises a shortened portion of the headband sized and dimensioned to couple with a second strap such that the second strap can slide along a length of the second strap coupling portion. In some embodiments, the headband comprises a first end portion and a second end portion, and a fastener for removably fastening the first end portion to the second end portion. The straps can be made of any suitable material(s), including for example, neoprene. In some aspects, the straps can comprise any suitable rubberized fabric, textile or material. In some aspects, the rubberized material can be water resistant or waterproof. In some aspects, the rubberized material can provide a non-slip or grip or friction capability to the substrate material. In some embodiments, the first strap comprises a first fastener component, and the second strap comprises a second fastener component complementary to the first fastener component. In some embodiments, the first strap coupling portion comprises a shortened portion of the headband sized and dimensioned to couple with the first strap such that the first strap can slide along a length of the first strap coupling portion. In some embodiments, the second strap coupling portion comprises a shortened portion of the headband sized and dimensioned to couple with the second strap such that the second strap can slide along a length of the second strap coupling portion. In some embodiments, the first strap is pivotable at the first strap coupling portion between 0-180 degrees. In some embodiments, the second strap is pivotable at the second strap coupling portion between 0-180 degrees. The first strap can comprise a looped portion sized and dimensioned to receive at least a portion of the first strap coupling portion, and the second strap can comprise a looped portion sized and dimensioned to receive at least a portion of the second strap coupling portion. In some embodiments, the first strap is rotatably coupled to the first strap coupling portion. In some embodiments, the second strap is rotatably coupled to the second strap coupling portion. In some embodiments, one or more of the straps can comprise openings for ventilation comfort/breathability.

### NON-LIMITING EMBODIMENTS

Embodiment 1. A headband configured to wrap around at least a portion of a head of a wearer, comprising: an inner side and an outer side; a first strap coupling portion; and a second strap coupling portion.
Embodiment 2. The headband of embodiment 1, wherein the first strap coupling portion comprises a shortened portion of the headband sized and dimensioned to couple with a first strap such that the first strap can slide along a length of the first strap coupling portion
Embodiment 3. The headband of any of embodiments 1-2, wherein the second strap coupling portion comprises a shortened portion of the headband sized and dimensioned to couple with a second strap such that the second strap can slide along a length of the second strap coupling portion.
Embodiment 4. The headband of any of embodiments 1-3, wherein the headband comprises a first end portion and a second end portion, and a fastener for removably fastening the first end portion to the second end portion.
Embodiment 5. The headband of any of embodiments 1-4, wherein the headband comprises a continuous loop, and wherein the headband is elastic enough to be placed on the head of the wearer and taken off the head of the wearer without a releasable fastener.
Embodiment 6. The headband of any of embodiments 1-5, wherein the headband comprises a neoprene material.
Embodiment 7. The headband of any of embodiments 1-6, wherein the headband is elastic.
Embodiment 8. The headband of any of embodiments 1-7, wherein the headband comprises a first band portion and a second band portion, wherein the first band portion is configured to be positioned above the second band portion.
Embodiment 9. The headband of any of embodiments 1-8, wherein the headband is pivotable at the first strap coupling portion between 0-180 degrees.
Embodiment 10. The headband of any of embodiments 1-9, wherein the headband is pivotable at the second strap coupling portion between 0-180 degrees.
Embodiment 11. A headband system, comprising: the headband of any of embodiments 1-10; a first strap; a second strap; and wherein the first strap is adjustably coupled to the first strap coupling portion, and wherein the second strap is adjustably coupled to the second strap coupling portion.
Embodiment 12. The system of embodiment 11, wherein the first strap comprises a looped portion sized and dimensioned to receive at least a portion of the first strap coupling portion.
Embodiment 13. The system of any of embodiments 11-12, wherein the second strap comprises a looped portion sized and dimensioned to receive at least a portion of the second strap coupling portion.
Embodiment 14. The system of any of embodiments 11-13, wherein the first strap comprises a first fastener.
Embodiment 15. The system of any of embodiments 11-14, wherein the second strap comprises a second fastener.
Embodiment 16. The system of any of embodiments 11-15, wherein the first strap is rotatably coupled to the first strap coupling portion.
Embodiment 17. The system of any of embodiments 11-16, wherein the second strap is rotatably coupled to the second strap coupling portion.
Embodiment 18. The system of any of embodiments 11-17, wherein the first strap comprises a set of openings for ventilation comfort.
Embodiment 19. The system of any of embodiments 11-18, wherein the second strap comprises a set of openings for ventilation comfort.
Embodiment 20. A cannula connection assembly for an air flow system cannula to be worn by a wearer having a cheek, comprising at least one of a tab and a strap configured to couple the cannula, wherein the at least one of the tab and the strap has a convex shape such that edge portions of the at least one of the tab and the strap face away from the cheek of the wearer when worn.
Embodiment 21. The cannula connection assembly of embodiment 20, wherein the at least one of the tab and the strap has a thickness of between 0.1-4 mm.
Embodiment 22. The cannula connection assembly of any of embodiments 20-21, wherein the cannula connection assembly consists of a single piece having a first end that couples to the cannula and a second end that couples to a headband.
Embodiment 23. The cannula connection assembly of any of embodiments 20-22, wherein the cannula connection assembly is made at least in part of silicone.
Embodiment 24. The cannula connection assembly of any of embodiments 20-23, wherein the cannula connection assembly is elastic.
Embodiment 25. The cannula connection assembly of any of embodiments 20-24, wherein the cannula connector assembly comprises a double tensioning mechanism from elasticity and an adjustable fastener.
Embodiment 26. The cannula connection assembly of any of embodiments 20-25, wherein the cannula connection assembly comprises the tab and the strap, and wherein the strap is adjustably coupled to at least one of the tab and a headband of any of embodiments 1-10.
Embodiment 27. The cannula connection assembly of any of embodiments 20-26, wherein the at least one of the tab and the strap comprises at least one of a set of cooling portions and a set of ventilation openings.
Embodiment 28. The cannula connection assembly of any of embodiments 20-27, wherein the at least one of the tab and the strap is angled to contour to the wearer's face.
Embodiment 29. A cannula apparatus, comprising: a nasal cannula; and a strap configured to wrap around a back of a wearer's head, and to couple to the air flow cannula.
Embodiment 30. The apparatus of embodiment 29, wherein the strap is configured to indirectly couple to the air flow cannula.
Embodiment 31. The apparatus of any of embodiments 29-30, wherein the strap is configured to indirectly couple to the air flow cannula via one or more cannula tabs.
Embodiment 32. The apparatus of any of embodiments 29-31, wherein the strap comprises a single band.
Embodiment 33. The apparatus of any of embodiments 29-32, wherein the strap is made at least in part of neoprene or a rubberized fabric.
Embodiment 34. The apparatus of any of embodiments 29-33, wherein the strap is made entirely of neoprene.
Embodiment 35. The apparatus of any of embodiments 29-34, wherein the strap comprises a fastener.
Embodiment 36. The apparatus of any of embodiments 29-35, wherein the strap comprises a first piece and a second piece, wherein the first piece has a first end portion and a second end portion, wherein the second piece has a third end portion and a fourth end portion, wherein the first end portion is configured to couple to at least one of the air flow cannula and a first tab, wherein the fourth end portion is configured to couple to at least one of the air flow cannula and a second tab, and wherein the second end portion and the third end portion are configured to removably fasten to one another via a first fastener.
Embodiment 37. The apparatus of any of embodiments 29-36, wherein the strap comprises a single piece having a first end portion and a second portion, wherein the first end portion is configured to couple to at least one of the air flow cannula and a first tab, and wherein the second end portion is configured to couple to at least one of the air flow cannula and a second tab.
Embodiment 38. A cannula apparatus including a headband, comprising: an air flow cannula; a first strap having a first end portion and a second end portion, the first end portion configured to couple to the air flow cannula, and the second end portion configured to couple to the headband; and a second strap having a third end portion and a fourth end portion, the third end portion configured to couple to the air flow cannula, and the fourth end portion configured to couple to the headband.
Embodiment 39. The cannula apparatus of embodiment 38, wherein each of the first strap and the second strap is configured to indirectly couple to the air flow cannula.
Embodiment 40. The cannula apparatus of any of embodiments 38-39, wherein each of the first strap and the second strap is configured to indirectly couple to the air flow cannula via one or more cannula tabs.
Embodiment 41. The cannula apparatus of any of embodiments 38-40, wherein the headband comprises any of the headbands of embodiments 1-10.
Embodiment 42. The cannula apparatus of any of embodiments 38-41, wherein each of the first strap and the second strap is made at least in part of neoprene.
Embodiment 43. The cannula apparatus of any of embodiments 38-42, wherein each of the first strap and the second strap is made entirely of neoprene.
Embodiment 44. The cannula apparatus of any of embodiments 38-43, wherein the first strap comprises a first fastener component, and the second strap comprises a second fastener component complementary to the first fastener component, and wherein at least one of the first strap and the second strap comprises two or more sections of different elasticity.
Embodiment 45. The cannula apparatus of any of embodiments 38-44, wherein the first strap coupling portion comprises a shortened portion of the headband sized and dimensioned to couple with the first strap such that the first strap can slide along a length of the first strap coupling portion.
Embodiment 46. The cannula apparatus of any of embodiments 38-45, wherein the second strap coupling portion comprises a shortened portion of the headband sized and dimensioned to couple with the second strap such that the second strap can slide along a length of the second strap coupling portion.
Embodiment 47. The cannula apparatus of any of embodiments 38-46, wherein the second strap is pivotable at the second strap coupling portion between 0-180 degrees, and wherein at least one of the first strap and the second strap can be removably coupled to any portion of the headband for maximum comfort.
Embodiment 48. The cannula apparatus of any of embodiments 38-47, wherein the first strap comprises a looped portion sized and dimensioned to receive at least a portion of the first strap coupling portion.
Embodiment 49. The cannula apparatus of any of embodiments 38-48, wherein the second strap comprises a looped portion sized and dimensioned to receive at least a portion of the second strap coupling portion.
Embodiment 50. The cannula apparatus of any of embodiments 38-49, wherein at least one of the first and second straps is configured to couple directly to the headband through via a fastener.
Embodiment 51. The cannula apparatus of any of embodiments 38-50, wherein the fastener is a hook and loop fastener.
Embodiment 52. The cannula apparatus of any of embodiments 38-51, wherein the first strap comprises a set of opening, and the second strap comprises a second set of openings for ventilation comfort.
Embodiment 53. The cannula apparatus of any of embodiments 38-52, wherein at least one of the first strap and the second strap comprises a set of cooling portions.
Embodiment 54. A tab configured to couple with a cannula and to adhere to at least one of a wearer's skin and a frame worn by the wearer.
Embodiment 55. The tab of embodiment 54, wherein the cannula tab is configured to adhere to the at least one of the wearer's skin and the frame worn by the wearer via an adhesive material.
Embodiment 56. The tab of any of embodiments 54-55, wherein the cannula tab is configured to adhere to the at least one of the wearer's skin and the frame worn by the wearer via negative pressure.
Embodiment 57. The tab of any of embodiments 54-56, wherein the frame comprises a nose frame.
Embodiment 58. The tab of any of embodiments 54-57, wherein the frame comprises a nose frame, and further comprising a fastener configured to adhere the cannula tab to the nose frame.
Embodiment 59. The tab of any of embodiments 54-58, wherein the fastener comprises a hook and loop fastener.
Embodiment 60. A nasal cannula, comprising at least one bellows section.
Embodiment 61. The nasal cannula of embodiment 60, wherein the at least one bellows section is positioned on a nasal prong.
Embodiment 62. A nasal cannula comprising at least one cooling portion.
Embodiment 63. A nasal cannula of any of embodiments 60-63, wherein the at least one cooling portion comprises a cooling gel material.
Embodiment 64. A tab for a cannula, the tab comprising a portion configured to contact a wearer's skin and comprising at least one cooling portion.
Embodiment 65. The tab of embodiment 64, wherein the at least one cooling portion comprises a cooling gel material.
Embodiment 66. A strap for a cannula, the strap comprising a portion configured to contact a wearer's skin and comprising at least one cooling portion.
Embodiment 67. The strap of embodiment 66, wherein the at least one cooling portion comprises a cooling gel material.
Embodiment 68. A headband configured to wrap around at least a portion of a head of a wearer, comprising: an inner side and an outer side, wherein at least one of the inner side and the outer side comprises a cooling portion; a first strap coupling portion; and a second strap coupling portion.
Embodiment 69. A nose frame coupled to a cannula, the nose frame comprising at least one cooling portion.
Embodiment 70. A patient interface assembly for a head of a wearer, comprising: a nasal cannula; a first connector extending from a first side of the nasal cannula; a second connector extending from a second side of the nasal cannula; a headband configured to wrap around at least a portion of the head of the wearer and maintain a position of the nasal cannula, the headband comprising: a first strap coupling portion and a second strap coupling portion, wherein the first strap coupling portion extends between a first stop and a second stop; a first strap having a first end portion and a second end portion, wherein the first end portion is coupled to the first strap coupling portion, wherein the first strap is movable along the first strap coupling portion between the first stop and the second stop, and wherein the second end portion is coupled to the first connector; and a second strap having a first end portion and a second end portion, wherein the first end portion is coupled to the second strap coupling portion, and wherein the second end portion is coupled to the second connector.
Embodiment 71. The assembly of embodiment 70, wherein the headband is pivotable at the first strap coupling portion.
Embodiment 72. The assembly of any of embodiments 70-71, wherein the first strap comprises a looped portion sized and dimensioned to receive at least a portion of the first strap coupling portion.
Embodiment 73. The assembly of any of embodiments 70-72, wherein the first strap comprises a set of openings for ventilation comfort.
Embodiment 74. The assembly of any of embodiments 70-73, wherein the first connector comprises a set of openings for ventilation comfort.
Embodiment 75. The assembly of any of embodiments 70-74, wherein the first strap comprises a cooling portion.
Embodiment 76. The assembly of any of embodiments 70-75, wherein the first connector comprises a cooling portion comprising a gel material.
Embodiment 77. The assembly of any of embodiments 70-76, wherein the nasal cannula comprises a tubular base having an inlet for receiving the flow of fluid, a first prong and a second prong, wherein each of the first prong and the second prong is fluidly connected to the inlet, and wherein the first prong comprises a flexible portion comprising a set of ridges and grooves.
Embodiment 78. A patient interface assembly for a head of a wearer, comprising: a nasal cannula; an elastic headband configured to wrap around at least a portion of the head of the wearer; a first connector extending from a first side of the nasal cannula; a second connector extending from a second side of the nasal cannula; and wherein the first connector and the second connector secure the nasal cannula to the elastic headband.
Embodiment 79. The assembly of embodiment 78, wherein each of the first connector and the second connector is made of a silicone.
Embodiment 80. The assembly of any of embodiments 78-79, wherein each of the first connector and the second connector comprises at least of a set of openings for ventilation comfort, and a cooling portion comprising a gel material.
Embodiment 81. The assembly of any of embodiments 78-80, wherein the first connector comprises a first end portion for removably coupling to a first portion of a headband via a first fastener.
Embodiment 82. The assembly of any of embodiments 78-81, wherein the second connector comprises a second end portion for removably coupling to a second portion of a headband via a second fastener.
Embodiment 83. The assembly of any of embodiments 78-82, wherein the first connector comprises a first end portion for removably coupling to a first strap via a first fastener, wherein the first strap is coupled to a first portion of a headband.
Embodiment 84. The assembly of any of embodiments 78-83, wherein the second connector comprises a second end portion for removably coupling to a second strap via a second fastener, wherein the second strap is coupled to a second portion of a headband.
Embodiment 85. The assembly of any of embodiments 78-84, wherein the nasal cannula comprises a tubular base having an inlet for receiving the flow of fluid, a first prong and a second prong, wherein each of the first prong and the second prong is fluidly connected to the inlet, and wherein the first prong comprises a flexible portion comprising a set of ridges and grooves.
Embodiment 86. A nasal cannula configured to deliver a flow of fluid to a user, the nasal cannula comprising: a tubular base having an inlet for receiving the flow of fluid; a first prong and a second prong, wherein each of the first prong and the second prong is fluidly connected to the inlet; and wherein the first prong comprises a set of grooves.
Embodiment 87. The nasal cannula of embodiment 86, wherein the first prong comprises a set of ridges.
Embodiment 88. The nasal cannula of any of embodiments 86-87, wherein the ridges of the set of ridges alternate with the grooves of the set of grooves.
Embodiment 89. The nasal cannula of any of embodiments 86-88, wherein the second prong comprises a second set of grooves.
Embodiment 90. The nasal cannula of any of embodiments 86-89, wherein the tubular base comprises a portion between the first and second prongs comprising a set of grooves.
Embodiment 91. The nasal cannula of any of embodiments 86-90, wherein the tubular base comprises a cooling portion made at least in part of a cooling gel material.

Thus, specific examples of headbands, straps, connectors (e.g., tabs), and other components for air flow systems have been disclosed. The above description of the disclosed embodiments is provided to enable any person skilled in the art to make or use the invention. The preceding detailed description is merely exemplary in nature and is not intended to limit the invention or the application and uses of the invention. Various modifications to these embodiments will be readily apparent to those skilled in the art, and the general principles described herein can be applied to other embodiments without departing from the spirit or scope of the invention. Thus, it is to be understood that the description and drawings presented herein represent a presently preferred embodiment of the invention and are therefore representative of the subject matter which is broadly contemplated by the present invention. It is further understood that the scope of the present invention fully encompasses other embodiments that may become obvious to those skilled in the art and that the scope of the present invention is accordingly not limited.

It will be understood that the benefits and advantages described above may relate to one embodiment or may relate to several embodiments. Aspects described in connection with one embodiment are intended to be able to be used with the other embodiments. Any explanation in connection with one embodiment applies to similar features of the other embodiments, and elements of multiple embodiments can be combined to form other embodiments. The embodiments are not limited to those that solve any or all of the stated problems or those that have any or all of the stated benefits and advantages.

The illustrations may include exaggerated dimensions and graphical representation to better illustrate the referenced items shown, and are not considered limiting unless expressly stated as such.

Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

As used herein and in the appended claims, the singular forms “a,” “an,” and “the” include plural referents unless the context clearly dictates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

Also, as used herein, and unless the context dictates otherwise, the term “coupled to” is intended to include both direct coupling (in which two elements that are coupled to each other contact each other) and indirect coupling (in which at least one additional element is located between the two elements).

Reference throughout this specification to “an embodiment” or “an implementation” or “some embodiments” means that a particular feature, structure, or characteristic described in connection with the embodiment is included in at least one embodiment or implementation. Thus, appearances of the phrases “in an embodiment” or “in some embodiments” in various places throughout this specification are not necessarily all referring to the same embodiment or a single exclusive embodiment. Furthermore, the particular features, structures, or characteristics described herein may be combined in any suitable manner in one or more embodiments or one or more implementations.

The word “exemplary” is used herein to mean “serving as an example, instance, or illustration.” Any aspect described herein as “exemplary” is not necessarily to be construed as preferred or advantageous over other aspects. Unless specifically stated otherwise, the term “some” refers to one or more.

Unless the context dictates the contrary, all ranges set forth herein should be interpreted as being inclusive of their endpoints and open-ended ranges should be interpreted to include only commercially practical values. Similarly, all lists of values should be considered as inclusive of intermediate values unless the context indicates the contrary.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Certain numerical values and ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating un-recited number may be a number which, in the context in which it is presented, provides the substantial equivalent of the specifically recited number.

Groupings of alternative elements or embodiments of the disclosure herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all groups used in the appended claims.

In the specification, reference may be made to the spatial relationships between various components and to the spatial orientation of various aspects of components as the devices are depicted in the attached drawings. However, as will be recognized by those skilled in the art after a complete reading of the present application, the devices, members, devices, etc. described herein may be positioned in any desired orientation. Thus, the use of terms such as “above,” “below,” “upper,” “lower,” “first”, “second” or other like terms to describe a spatial relationship between various components or to describe the spatial orientation of aspects of such components should be understood to describe a relative relationship between the components or a spatial orientation of aspects of such components, respectively, as the device described herein may be oriented in any desired direction.

Combinations, described herein, such as “at least one of A, B, or C,” “one or more of A, B, or C,” “at least one of A, B, and C,” “one or more of A, B, and C,” and “A, B, C, or any combination thereof” include any combination of A, B, and/or C, and may include multiples of A, multiples of B, or multiples of C. Specifically, combinations such as “at least one of A, B, or C,” “one or more of A, B, or C,” “at least one of A, B, and C,” “one or more of A, B, and C,” and “A, B, C, or any combination thereof” may be A only, B only, C only, A and B, A and C, B and C, or A and B and C, and any such combination may contain one or more members of its constituents A, B, and/or C. For example, a combination of A and B may comprise one A and multiple B's, multiple A's and one B, or multiple A's and multiple B's.

All structural and functional equivalents to the components of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. A patient interface assembly for a head of a wearer, comprising:
a nasal cannula;
a first connector extending from a first side of the nasal cannula;
a second connector extending from a second side of the nasal cannula;
a headband configured to wrap around at least a portion of the head of the wearer and maintain a position of the nasal cannula, the headband comprising:
a first strap coupling portion and a second strap coupling portion, wherein the first strap coupling portion extends between a first stop and a second stop;
a first strap having a first end portion and a second end portion, wherein the first end portion is coupled to the first strap coupling portion, wherein the first strap is movable along the first strap coupling portion between the first stop and the second stop, and wherein the second end portion is coupled to the first connector; and
a second strap having a first end portion and a second end portion, wherein the first end portion is coupled to the second strap coupling portion, and wherein the second end portion is coupled to the second connector.

2. The patient interface assembly of claim 1, wherein the headband is pivotable at the first strap coupling portion.

3. The patient interface assembly of claim 1, wherein the first strap comprises a looped portion sized and dimensioned to receive at least a portion of the first strap coupling portion.

4. The patient interface assembly of claim 1, wherein the first strap comprises a set of openings for ventilation comfort.

5. The patient interface assembly of claim 1, wherein the first connector comprises a set of openings for ventilation comfort.

6. The patient interface assembly of claim 1, wherein the first strap comprises a cooling portion.

7. The patient interface assembly of claim 1, wherein the first connector comprises a cooling portion comprising a gel material.

8. The patient interface assembly of claim 1, wherein the nasal cannula comprises a tubular base having an inlet for receiving the flow of fluid, a first prong and a second prong, wherein each of the first prong and the second prong is fluidly connected to the inlet, and wherein the first prong comprises a flexible portion comprising a set of ridges and grooves.

9. A patient interface assembly for a head of a wearer, comprising:
a nasal cannula;
an elastic headband configured to wrap around at least a portion of the head of the wearer;
a first connector extending from a first side of the nasal cannula;
a second connector extending from a second side of the nasal cannula; and
wherein the first connector and the second connector secure the nasal cannula to the elastic headband.

10. The patient interface assembly of claim 9, wherein each of the first connector and the second connector is made of a silicone.

11. The patient interface assembly of claim 9, wherein each of the first connector and the second connector comprises at least of a set of openings for ventilation comfort, and a cooling portion comprising a gel material.

12. The patient interface assembly of claim 9, wherein the first connector comprises a first end portion for removably coupling to a first portion of a headband via a first fastener.

13. The patient interface assembly of claim 12, wherein the second connector comprises a second end portion for removably coupling to a second portion of a headband via a second fastener.

14. The patient interface assembly of claim 9, wherein the first connector comprises a first end portion for removably coupling to a first strap via a first fastener, wherein the first strap is coupled to a first portion of a headband.

15. The patient interface assembly of claim 14, wherein the second connector comprises a second end portion for removably coupling to a second strap via a second fastener, wherein the second strap is coupled to a second portion of a headband.

16. The patient interface assembly of claim 9, wherein the nasal cannula comprises a tubular base having an inlet for receiving the flow of fluid, a first prong and a second prong, wherein each of the first prong and the second prong is fluidly connected to the inlet, and wherein the first prong comprises a flexible portion comprising a set of ridges and grooves.

17. A nasal cannula configured to deliver a flow of fluid to a user, the nasal cannula comprising:
a tubular base having an inlet for receiving the flow of fluid;
a first prong and a second prong, wherein each of the first prong and the second prong is fluidly connected to the inlet; and
wherein the first prong comprises a set of grooves.

18. The nasal cannula of claim 17, wherein the first prong comprises a set of ridges.

19. The nasal cannula of claim 18, wherein the ridges of the set of ridges alternate with the grooves of the set of grooves.

20. The nasal cannula of claim 17, wherein the second prong comprises a second set of grooves.

21. The nasal cannula of claim 17, wherein the tubular base comprises a portion between the first and second prongs comprising a set of grooves.

22. The nasal cannula of claim 17, wherein the tubular base comprises a cooling portion made at least in part of a cooling gel material.
